(19) United States
(12) Patent Application Publication
(10) Pub. No.: US 2011/0087291 A1
(43) Pub. Date: Apr. 14, 2011

(54) FUSION IMPLANTS AND SYSTEMS FOR POSTERIOR LATERAL PROCEDURES

(75) Inventors: Jeff R. Justis, Germantown, TN (US); Hai H. Trieu, Cordova, TN (US)

(73) Assignee: Warsaw Orthopedic, Inc., Warsaw, IN (US)

(21) Appl. No.: 12/578,896

(22) Filed: Oct. 14, 2009

(51) Int. Cl. A61B 17/70; A61B 17/88

(52) U.S. Cl. 606/264; 606/279

(57) ABSTRACT

Apparatus and methods include implants insertable into the body of a patient for posterior lateral fusion of one or more vertebral levels. The implant can be osteoinductive and/or osteoconductive to facilitate or initiate the fusion between two or more bony portions of the spinal column. The fusion implant can be used in isolation or in conjunction with a posterior stabilization construct.

### BACKGROUND

The invention relates generally to medical devices and procedures. More particularly, the invention relates to apparatus and methods for fusion in posterior lateral procedures, and in procedures at other locations associated with the spine.

Stabilization of one or more levels of the spine is often accomplished with placement of a rod construct between bone anchors engaged to the vertebrae of the one or more levels. These procedures may also involve placement of an implant and/or bone graft material in the disc space between the vertebrae to support fusion of the vertebrae. Insertion and placement of the implant and/or bone graft material employs an approach for entry into the patient that is separated from or greater in size than that required for placement of the rod and bone anchors. Therefore, the invasiveness of the procedure is increased.

Thus, a need exists for improved implants and procedures for insertion and securement of implants at locations within a patient's body that can minimize intrusion and invasiveness into tissue of the patient, reducing post-operative pain and healing time for the patient.

### SUMMARY

Apparatus, methods, systems, procedures and implants for fusion of one or more levels of the spine are described herein. In some embodiments, the systems includes a stabilization construct to stabilize one or more vertebral levels and a fusion implant positioned adjacent the stabilization construct between bony portions of the vertebrae outside the spinal disc space between the vertebrae. In other embodiments, the methods include placement of an elongate connecting member between bone anchors engaged to vertebrae and placement of a fusion implant between bony portions of the vertebrae along a minimally invasive insertion path that generally parallels the insertion path of the connecting member. In other embodiment, an implant system includes an elongate connecting portion positionable between and engageable to anchors engaged to vertebrae and a fusion portion deployable from the connecting portion after insertion of the connecting portion to extend between and connect bony portions of the vertebrae. The apparatus, methods, systems, procedures and implants minimize invasiveness of the procedure into the patient. Applications in non-minimally invasive procedures are also contemplated.

According to a further aspect, an implant system includes at least a first anchor engageable to a first vertebra and a second anchor engageable to a second vertebra. The system further includes an elongate connecting member extending along an axis between a first end and an opposite second end with a length between the first and second ends sized to extend between and be engaged to the first and second anchors when the first and second anchors are engaged to the first and second vertebrae. The implant system also includes an elongate fusion member extending between a first end and an opposite second end. The elongate fusion member is comprised of bone growth material and includes a length between the first and second ends sized for positioning in contact with bony portions of the first and second vertebrae when the elongate fusion member is positioned alongside the connecting member.

According to another aspect, an implant system includes at least a first anchor engageable to a first vertebra and a second anchor engageable to a second vertebra. The implant system also includes an elongate stabilization member including a connection portion extending along a longitudinal axis between a first end and an opposite second end. The connection portion includes a length between the first and second ends sized to extend between and be engaged to the first and second anchors when the first and second anchors are engaged to the first and second vertebrae. The elongate stabilization member further includes a deployable fusion portion carried by the connection portion. The fusion portion includes a first collapsed condition for insertion of the elongate stabilization member between the first and second anchors and a second deployed condition projecting outwardly from the connection portion sized to engage bony portions of the first and second vertebrae. The fusion portion includes an interior with bone growth material in the interior in the deployed condition to facilitate fusion of the bony portions of the first and second vertebrae.

According to yet another aspect, a method comprises: inserting through an incision in a patient an elongate connecting member between first and second anchors engaged to posterior elements of the first and second vertebrae; engaging the connecting member to the first and second anchors; inserting a fusion member through the incision to a location adjacent to the first and second anchors and the connecting member and positioning the fusion member in contact with bony portions of the first and second vertebrae; and engaging the fusion member to at least one of the first and second anchors and transverse processes of the first and second vertebrae.

These and other aspects are also discussed below.

### BRIEF DESCRIPTION OF THE DRAWINGS

FIG. 1 is an elevation view of a posterior portion of a spinal column motion segment with an implant system engaged thereto.

FIG. 2 a lateral view of the spinal column motion segment and implant system of FIG. 1.

FIG. 3 is an elevation view of another embodiment implant system positioned between anchors and transverse processes of the spinal column motion segment.

FIG. 4 is the implant of FIG. 3 with a deployed fusion portion of the implant engaged to the transverse processes of the spinal column motion segment.

FIG. 5 is a section view along line 5-5 of FIG. 4.

FIG. 6 is an elevational view of an insertion instrument and implant construct engaged to a diagrammatically shown spinal motion segment.

FIG. 7 an elevational view of the insertion instrument and stabilization construct of

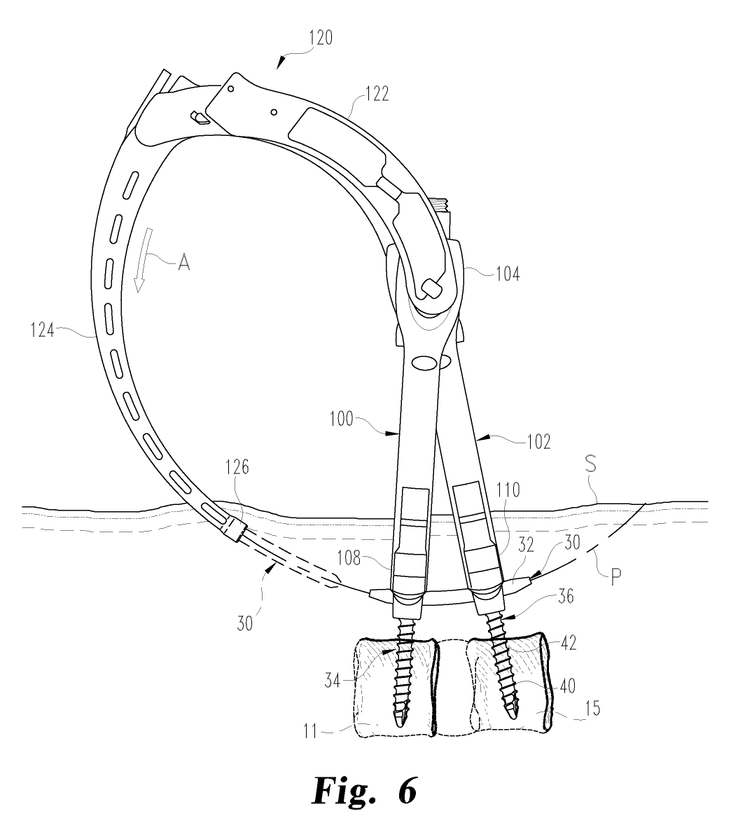

FIG. 6 and a fusion implant engaged to a fusion implant inserter positioned along an approach corridor to the spinal motion segment that generally parallels the approach of the insertion instrument to the spinal motion.

FIG. 8 is an endwise elevational view showing the relative positions between the stabilization construct and fusion implant when implanted.

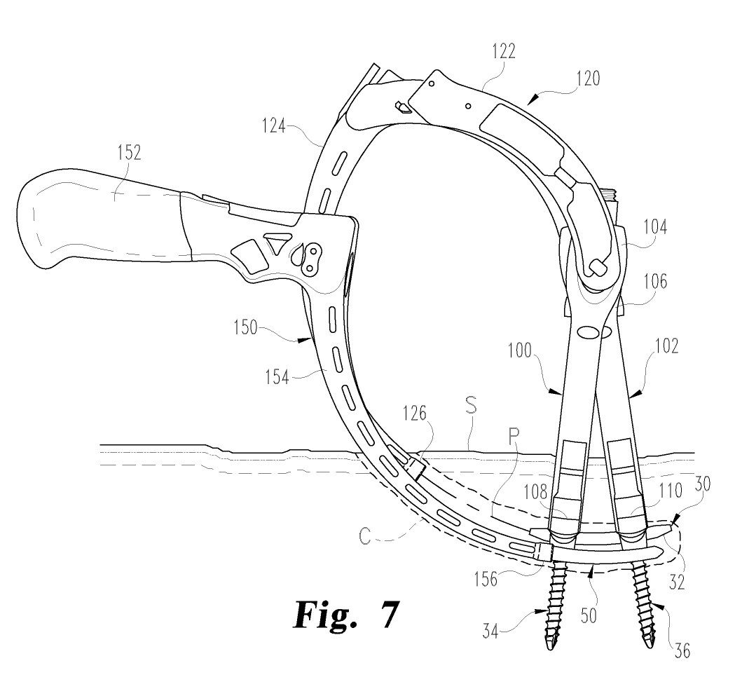

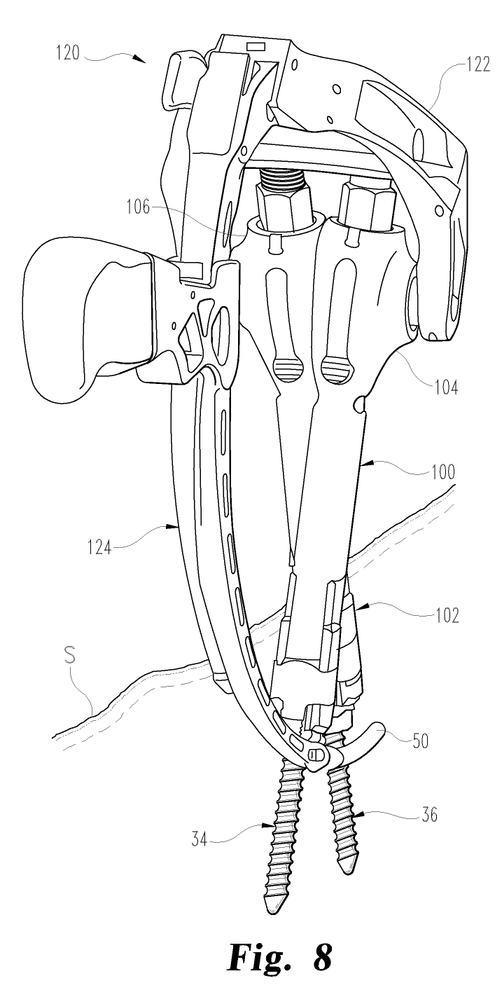

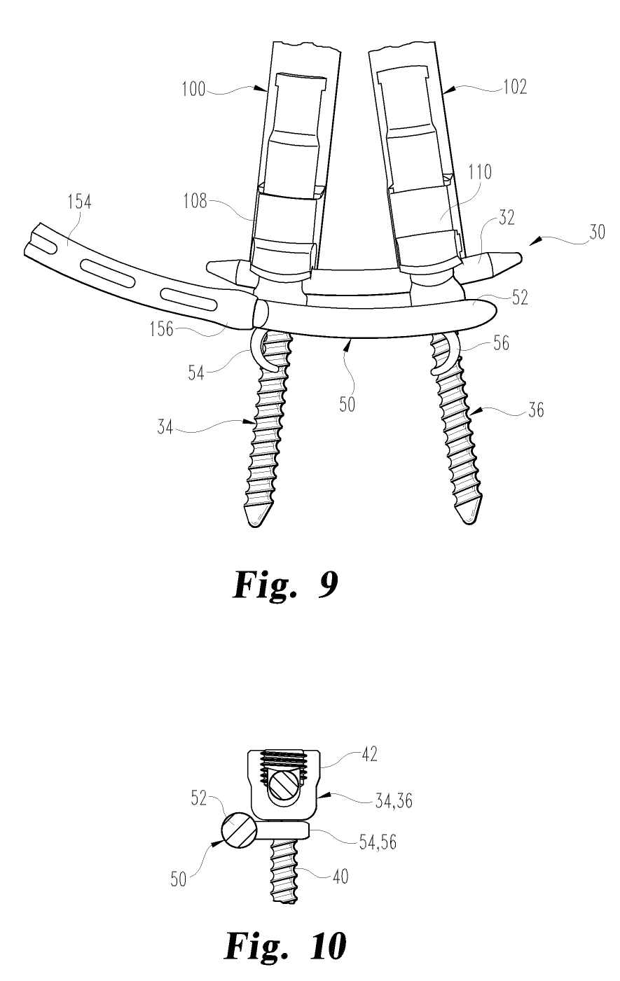

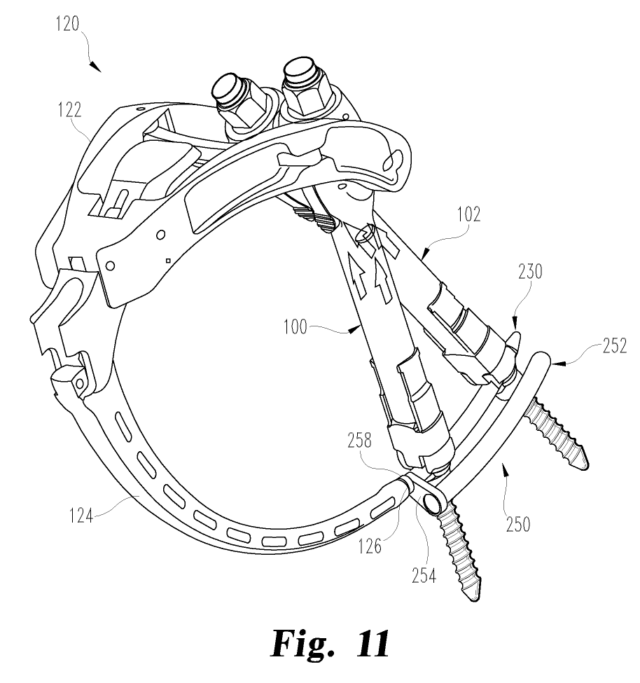

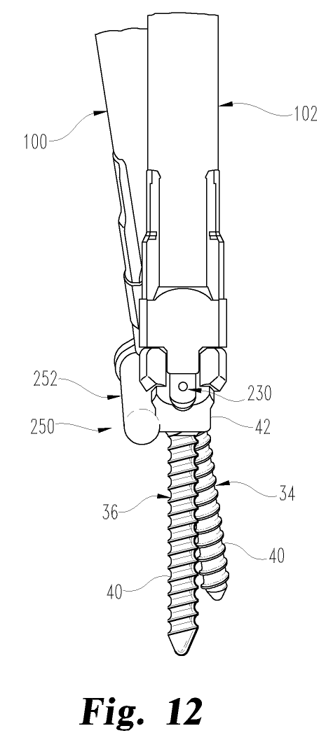

FIG. 9 is an enlarged elevational view of the stabilization construct and fusion implant of FIGS. 7-8 showing deployment of engagement features of the fusion implant to engage transverse processes of vertebrae of the spinal column motion segment.

FIG. 10 is an end elevation view showing an alternate arrangement of the fusion implant with engagement features deployed to engage anchors of the stabilization construct.

FIG. 11 is a perspective view showing another embodiment fusion implant engaged to the insertion instrument of the stabilization construct.

FIG. 12 is an end elevation view showing the relative positions between the fusion implant and stabilization construct of FIG. 11.

### DETAILED DESCRIPTION

For the purposes of promoting an understanding of the principles of the invention, reference will now be made to the embodiments illustrated in the drawings and specific language will be used to describe the same. It will nevertheless be understood that no limitation on the scope of the invention is intended. Any alterations and further modifications in the illustrated devices and described methods and further applications of the principles of the invention as disclosed herein are contemplated as would normally occur to one skilled in the art to which the invention relates.

Apparatus and methods include implant systems insertable into the body of a patient for stabilization and fusion along, for example, posterior lateral portions of two or more vertebrae. The fusion implant can be osteoinductive and/or osteoconductive to facilitate or initiate the fusion between two or more bony portions of the spinal column. The fusion implant can be used in isolation or in conjunction with a posterior stabilization construct. In one embodiment, the stabilization construct includes at least two anchors engageable to respective ones of first and second vertebrae and an elongated connecting member positioned between and engaged to the at least two anchors. The fusion implant is positioned along the stabilization construct and between bony portions of the first and second vertebrae for fusion therebetween. The fusion implant is engageable to at least one or both of the stabilization construct and the bony portions when implanted. In one embodiment, the fusion implant includes engagement features that project outwardly from an elongated body of the fusion implant to engage at least one or both of the stabilization construct and the bony portions. In another embodiment, the stabilization system includes an elongate connecting portion extending between and engaged to the anchors and a fusion portion deployable from the connecting portion into the space between bony portions of the vertebrae. Other embodiments contemplate connecting members and/or fusion implants that extend between or are positioned between three or more anchors engaged to three or more vertebrae.

In some embodiments, a method includes inserting percutaneously or through a small incision at least first and second anchors and engaging the first and second anchors to respective ones of first and second bony portions. The method further includes guiding an elongated connecting member into the patient along a minimally invasive insertion path and between the anchors, and engaging the connecting member to the first and second anchors to stabilize the first and second bony portions. The method also includes guiding a fusion implant along generally the same minimally invasive insertion path and engaging the fusion implant directly to the first and second bony portions, or alternatively or additionally, engaging the fusion implant directly to the stabilization construct. The stabilization construct can be structured with an elongate connecting member engaged to the first and second anchors to provide completely rigid, semi-rigid, or flexible stabilization of the bony portions during fusion. The fusion implant can be structured as a second elongate member that is positioned adjacent to the first elongate connecting member, or as a fusion portion that is deployable from the elongate connecting member.

In some embodiments, a method includes elongate extensions extending proximally from first and second anchors engaged to first and second bony portions of vertebrae, mounting an inserter to a proximal end portion of at least one of the elongate extensions, and guiding an elongate connecting member to the first and second anchors by moving the inserter relative to the at least one extension. A fusion member is then mounted to the inserter and inserted to a location adjacent to the connecting member and into contact with bony portions of the vertebrae. Alternatively, the fusion member can be mounted to a second inserter instrument and guided along an insertion path that generally follows the insertion path of the connecting member to a location adjacent the connecting member. In a further alternative, the connecting member and fusion member are inserted simultaneously along the minimally invasive insertion path. The fusion member includes engagement features that are then engaged to the first and second bony portions, the first and second anchors, or both the bony portions and the first and second anchors.

In other embodiments, a method includes guiding an elongate connecting member to a location between first and second anchors and engaging the connecting member to the first and second anchors. A fusion portion is then deployed from the connecting member into the space between and into contact with the first and second bony portions.

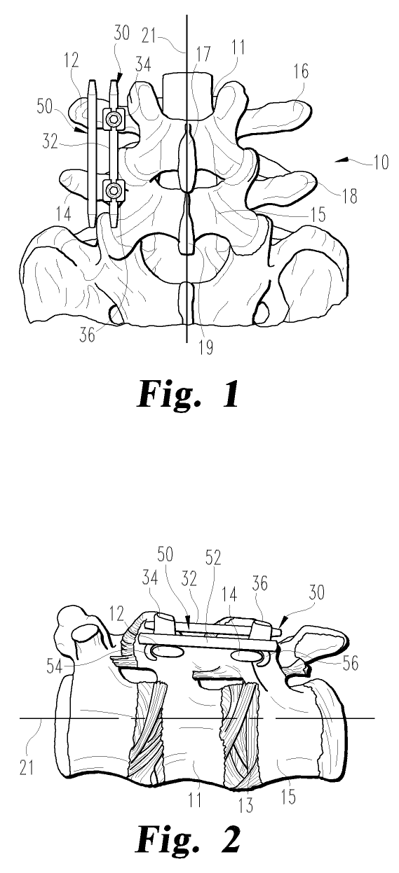

In FIGS. 1-2 there is shown a spinal column segment 10 with a vertebral level including an upper vertebra 11, a disc 13, and a lower vertebra 15 extending along a central axis 21 of the spinal column. The vertebrae 11, 15 and the disc 13 therebetween comprise a vertebral level of a spinal motion segment, it being understood that a spinal motion segment may include one or more vertebral levels. Upper vertebra 11 includes a first upper transverse process 12 and a second upper transverse process 16. Lower vertebra 15 includes a first lower transverse process 14 and a second lower transverse process 18. The transverse processes 12, 14, 16, 18 comprise posterior elements of the vertebrae of the spinal motion segment along with the spinous processes 17, 19, facets, pedicles and other posterior structures of each vertebrae 11, 15. It is contemplated that the spinal column segment is part of a patient in which spinal surgery is to be performed with the present invention. It is also contemplated that the spinal column segment may comprise a non-human or non-living animal substrate, such as may be present with a training model to teach methods employing the surgical instruments and implants discussed herein.

A stabilization construct 30 is positioned in engagement with the posterior vertebral elements, such as the pedicles of vertebrae 11, 15, to provide spinal stabilization. Stabilization construct 30 includes a first elongate connecting member 32 extending between and engaged to anchors 34, 36 secured to vertebrae 11, 15. First connecting member 32 provides stabilization of vertebrae 11, 15 to limit or prevent motion of the spinal motion segment. It is further contemplated that stabilization construct 30 can be extended to one or more additional vertebrae, or that one or more additional spinal stabilization constructs 30 can be engaged to the contra-lateral side of the spinal motion segment and/or along or more additional vertebral levels. In addition, a fusion implant 50 is positioned adjacent to stabilization construct 30 and engaged to transverse processes 12, 14. Fusion implant 50 includes an elongated body 52 extending between transverse processes 12, 14. As discussed further below, elongated body 52 includes engagement features 54, 56 that directly engage transverse processes 12, 14 and/or directly engage stabilization construct 30 to maintain the positioning of fusion implant 50 during fusion of the transverse processes 12, 16.

Various configurations for fusion implant 50 are contemplated. In one embodiment, fusion implant 50 includes an osteoinductive body 52 extending between opposite leading and trailing ends of the body, and the body is comprised of material that induces bone growth to fuse vertebrae 11, 15 between transverse processes 12, 16. In another embodiment, fusion implant 50 includes an osteoconductive body 52 that is comprised of material that conducts bone growth to fuse vertebrae 11, 15 between transverse processes 12, 16. In still other embodiment, fusion implant 50 includes an elongate body 52 comprising a mesh bag packed with bone material or bone substitute material to support fusion. Fusion implant 50 is positioned laterally adjacent to connecting member 32 and in contact with transverse processes 12, 16 of vertebrae 11, 15. Engagement features 54, 56 include hooks, spikes, collars, clamps or other structure to secure body 52 to the transverse processes 12, 16 or to stabilization construct 30.

In one particular embodiment, engagement features 54, 56 are comprised of shape memory material and initially are configured to extend along or be housed within body 52 prior to implantation so that fusion implant 50 can be positioned into the desired location along a minimally invasive insertion path to the implantation location without engagement features 54, 56 substantially interfering with the insertion. After implantation, the temperature of the patient's body causes the shape memory material to react and deploy engagement features 54, 56 from the insertion configuration and into their implanted configuration into engagement with the transverse processes 12, 16 or stabilization construct 30 to maintain the implanted position of fusion implant 50.

Other embodiments contemplate that engagement features 54, 56 are deployed by manipulating engagement features 54, 56 with a tool, or by altering the length, width, curvature or other feature of body 52 of fusion implant 50. In still other embodiments, engagement features 54, 56 are separately attached to body 52 after body 52 is positioned in the patient, or engagement features 54, 56 are secured to the desired structure in the patient and body 52 is then mounted to engagement features 54, 56 upon insertion of fusion implant 50 into the body.

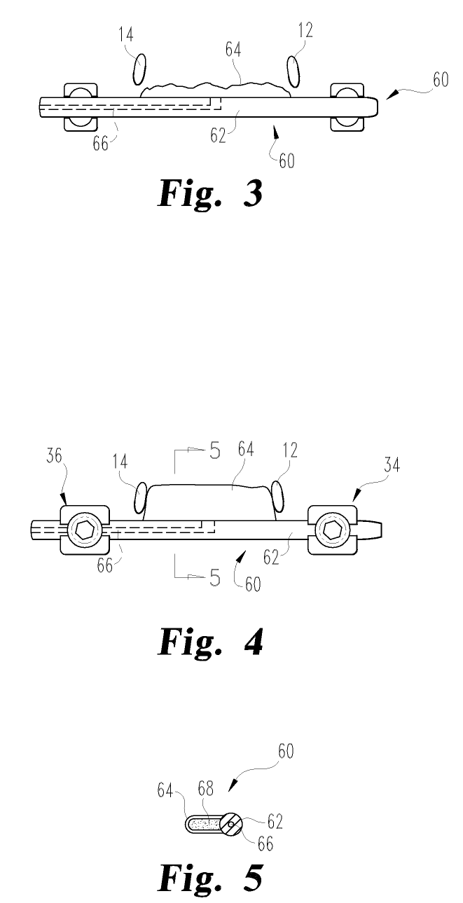

FIGS. 3-5 show another embodiment implant 60 in which the elongate connecting member of the stabilization construct incorporates the fusion implant as a part thereof. Implant 60 includes an elongate connecting portion 62 that is configured to extend between and be engaged to anchors 34, 36. Elongate connecting portion 62 provides a desired stabilization effect of the bony portions to which anchors 34, 36 are engaged. For example, connecting portion 62 can provide rigid stabilization to prevent motion of the bony portions, or semi-rigid or flexible stabilization of the bony portions. Fusion implant 60 also includes a fusion portion 64 that is carried by connecting portion 62. In one embodiment, fusion portion 64 is a collapsible bag, balloon, or other type of housing that can be reduced in size to facilitate minimally invasive insertion of fusion implant 60, and then increased in size after insertion so that fusion portion 64 extends between and contacts the bony portions.

In FIG. 3 implant 60 is positioned with connecting portion 62 between anchors 34, 36, and with fusion portion 64 positioned toward transverse processes 12, 16. Fusion portion 64 is in an undeployed or collapsed condition to facilitate insertion of implant 60 into the patient. Connecting portion 62 is engaged to anchors 34, 36 in FIG. 4, and fusion portion 64 is deployed to project outwardly from connecting portion 62 and into contact with transverse processes 12, 16. In the deployed position, fusion portion 64 extends along and contacts at least a portion of transverse processes 12, 16 to provide a platform for fusion of the bony portions between transverse processes 12, 16.

Connecting portion 62 includes a lumen 66 extending axially therealong that is in fluid communication with an interior of fusion portion 64. Bone growth material 68 can be delivered through lumen 66 to fusion portion 64 to expand or deploy fusion portion 64. Fusion portion 64 can be housed completely within connecting portion 62 prior to deployment, or carried alongside connecting portion 62 in an undeployed or collapsed configuration during insertion into the patient. Fusion portion 64 can include attachment mechanisms at one or both of the ends of fusion portion 64 that attaches fusion portions to transverse processes 12, 14, the bone anchors 34, 36, and/or connecting member 32. Examples of suitable attachment mechanisms include hooks, spikes, caps, anchors, and teeth, for example.

Fusion portion 64 can be made from mesh material, permeable or semi-permeable material, porous material, restorable material, semi-resorbable material, or permanent material. In one embodiment, the material allows bone growth therethrough. Examples of suitable material for fusion portion 64 include woven fabric tubing, woven and non-woven mesh, or braided or woven structures, and folded woven fabric. Additionally, fusion portion 64 may be resilient and/or elastic so it can assume various shapes during and after insertion and attachment. Growth factors or cells can be incorporated into fusion portion 64 and/or bone growth material 68 to accelerate the bone growth process. Growth factors can be transforming growth factor 81, insulin-like growth factor 1, platelet-derived growth factor, fibroblast growth factor, bone morphogenetic protein (BMP), LIM mineralization protein (LMP) and combinations thereof.

Fusion portion 64 can be made from any biocompatible material, material of synthetic or natural origin, and material of a resorbable or non-resorbable nature. Examples of suitable resorbable materials including polylactide, polyglycolide, tyrosine-derived polycarbonate, polyanhydride, polyorthoester, polyphosphazene, calcium phosphate, hydroxyapatite, bioactive glass, collagen, albumin, fibrinogen and combinations thereof; and example of suitable non-resorbable materials include polyethylene, polyester, polyvinyl alcohol, polyacrylonitrile, polyamide, polytetrafluorethylene, poly-paraphenylene terephthalamide, cellulose, and combinations thereof.

FIGS. 6-9 show one embodiment insertion technique and instrumentation for implanting the stabilization construct and fusion implant. In FIG. 6, vertebrae 11, 15 are shown diagrammatically and positioned below skin level S with tissue of the patient between the vertebrae and skin S. Anchors 34, 36 are engaged to vertebrae 11, 15. Anchors 34, 36 each include a bone engaging portion 40 and a receiving portion 42 mounted to bone engaging portion 40. In the illustrated embodiment, bone engaging portion is a bone screw and receiving portion 42 is a saddle or U-shaped head pivotally mounted to the head of the bone screw. Receiving portion 42 is movable to rotate around the head of the bone screw to align passages of the receiving portions 42 to receive connecting member 32. The passages of receiving portions 42 open proximally to receive a set screw, cap or other engaging member to secure elongate member in receiving portions 42. Other embodiments contemplate receiving portions with laterally opening or obliquely opening passages, or passages that are encircled by receiving portion 42. In any event, the receiving portions 42 open at the cephaladly and caudally oriented ends of receiving portions 42 to accept connecting member 32 in an endwise manner from an insertion path P so that a leading end of connecting member 32 passes through anchor 34 and then anchor 36 and an opposite trailing end of connecting member 32 is located adjacent to anchor 34. Bone engaging portions 40 are shown as bone screws, but can also include any suitable bone engagement structure, including hooks, staples, spikes, bolts, wires, or clamps, for example.

The insertion instrumentation includes anchor extensions 100, 102 extending proximally from respective ones of anchors 34, 36 through skin level S to proximal end portions 104, 106. Extensions 100, 102 include distal end portions 108, 110, respectively, removably engaged to respective ones of anchors 34, 36. The insertion instrumentation further includes an inserter 120 mounted to proximal end portions 104, 106 of extensions 100, 102. Inserter 120 includes a mounting portion 122 movably mounted to extensions 100, 102, and an elongate arm 124 extending transversely from mounting portion 122 for movement along an arc A that parallels insertion path P. Connecting member 32 is removably engageable to the distal end 126 of elongate arm 124 and movable therewith along insertion path P through skin S and tissue of the patient from a location outside the patient toward anchors 34, 36 and then between anchors 34, 36 for engagement thereto. Additional features and embodiments of anchor extensions and inserters are provided in U.S. Pat. No. 6,530,929 issued Mar. 11, 2003; U.S. Pat. No. 7,188,626 issued Mar. 13, 2007; U.S. Patent App. Pub. No. 2005/0171540 published on Aug. 4, 2005; U.S. Patent App. Pub. No. 2007/0049931 published on Mar. 1, 2007; and U.S. Patent App. Pub. No. 2008/0319477 published on Dec. 5, 2008; each of which is incorporated herein by reference in its entirety.

FIGS. 7-8 show a second inserter 150 positioned through the same incision in skin S as arm 124 of inserter 120. Second inserter 150 includes a proximal handle 152 and a distal arm 154 extending transversely to handle 152 along an arc that generally corresponds to the arc along which arm 124 extends. This facilitates positioning fusion implant 150 along an insertion path that follows or at least generally follows insertion path P of elongate member 32. Fusion implant 50 is removably engaged to the distal end 156 of arm 154 of second inserter 150. Fusion implant 50 is guided to a location laterally adjacent to anchors 34, 36 so that fusion implant 50 is located distally of, or more anteriorly toward, vertebrae 11, 15, than elongate member 32. This positions fusion implant 50 in contact with the transverse processes 12, 16 to support fusion between transverse processes 12, 14. In the implanted configuration, connecting member 32 and fusion implant 50 are located in laterally and distally offset relation to one another, and extend generally parallel to one another while being spaced a distance from one another.

Second inserter 150 allows insertion of fusion implant 50 generally along pathway P using a freehand technique. Arm 124 of inserter 120 can remain positioned in the opening through skin S and tissue along which connecting member 32 was inserted to provide retraction and initial guidance regarding the placement of the leading end of fusion implant 50. The surgeon can monitor insertion and positioning of fusion implant 50 using fluoroscopy or other suitable imaging system. Additional details and embodiments of second inserter 150 are disclosed in U.S. Pat. No. 7,520,879 issued Apr. 21, 2009, which is incorporated herein by reference in its entirety. It is further contemplated that connecting member 32 can be implanted with a freehand technique employing second inserter 150.

In the implanted position, fusion implant 50 is located so that body 52 is positioned adjacent to bone engaging portions 40 of anchors 34, 36 just below receiving portions 42. The insertion paths of fusion implant 50 and connecting member 30 extend along a minimally invasive corridor C through the tissue of the patient to the implanted position. In one embodiment, corridor C is curved from skin S to the implanted position of the implant system. Once fusion implant 50 is in the desired position, engagement features 54, 56 are deployed as shown in FIG. 9 to engage, for example, the transverse processes 12, 16. In another embodiment, shown in FIG. 10, engagement features 54, 56 are oriented laterally toward anchors 34, 36 and deployed to engage, for example, bone engaging portions 40 of the stabilization construct. Engagement features 34, 36 engage the desired structure to maintain the positioning of fusion implant 50 between the bony portions of transverse processes 12, 16.

FIGS. 11-12 show another embodiment implant system 250 that includes an elongate connecting member 230 and an elongate fusion member 252 that can each be coupled to distal end 126 of inserter arm 124. Implant system 250 further includes a connector 254 extending laterally from inserter arm 124 and engaged to the trailing end of fusion member 252 so that fusion member 252 is laterally offset from connecting member 230 when implanted with inserter 120. Elongate connecting member 230 is positioned between anchors 34, 36 with inserter 120 in the manner discussed above for connecting member 30, and then fusion member 252 with connector 254 is engaged to inserter 120 and positioned into the patient along an insertion path the generally parallels insertion path P for elongate connecting member 230. In another embodiment, connector 254 couples connecting member 230 and fusion member 252 to one another so that they are simultaneously inserted into the patient along path P toward anchors 34, 36.

Connector 254 includes an extension 258 that is axially aligned with the trailing end of connecting member 230. Extension 258 is received in and gripped by inserter 120 at the distal end 126 of arm 124. Extension 258 extends from connector 254 in a direction away from connecting member 230. Furthermore, connector 254 extends laterally from distal end 126 and the trailing end of first member 230 so that fusion member 252 is offset a distance from connecting member 230 along the length of fusion member 252. Fusion member 252 is further offset below or toward the bony portions from connecting member 230 so that fusion member 252 is positioned along the distal sides of receiving portions 42 with elongate connecting member 230 is positioned through receiving portions 42.

In one embodiment, the fusion implant or fusion portions are comprised at least partially of bone growth promoting material. Examples of suitable bone growth material include a bone morphogenic protein (BMP). However, other types of bone growth promoting materials are also contemplated for use in association with the fusion implant and fusion portion, such as, for example, a bone graft material including autograft, allograft, xenograft, bone chips, bone marrow, demineralized bone matrix (DBM), mesenchymal stem cells, LIM mineralization protein (LMP), rh-BMP2, rh-BMP7, TGF-beta, platelet-derived growth factors, synthetic bone graft, calcium phosphate, hydroxyaptite, calcium carbonate, bioactive glass, or any other suitable bone growth promoting material. Additionally, it should be understood that the bone growth promoting material may be used with or without a suitable carrier. In addition, these materials may be designed and/or processed to include shape memory capabilities to deploy engagement features from the fusion implant or fusion portion via material transition by temperature change, removal or imposition of force, or other suitable deployment technique. Furthermore, the engagement features may be made from the same material, or from different material, than the fusion portion or fusion implant.

It is further contemplated that the connecting member or connecting portion extending between and engaged to the anchors can be comprised of a metal material, such as stainless steel, titanium, chrome-cobalt alloys. The connecting member or connecting portion may also be comprised of a polymer, such as, for example, polyetheretherketone (PEEK), polyetherketoneketone (PEKK), polymethylmethacrylate, polyurethane, silicone, silicone-polyurethane copolymers, epoxy, polycarbonate, polyketone, polyester, polyethylene, polyimide, polylactic acid, polypropylene, polystyrene, polysulfone, polyvinyl chloride, polyamide, poly(tetrafluoroethene), polyphthalamide, polybutylene and mixtures or combinations of thereof. The connecting member or connecting portion and/or anchors may also be comprised of suitable resorbable material as identified herein and resorbable over time such that the stabilization construct is resorbed after fusion of the bony portion.

Although various embodiments have been described as having particular features and/or combinations of components, other embodiments are possible having a combination of any features and/or components from any of embodiments as discussed above. As used in this specification, the singular forms “a,” “an” and “the” include plural referents unless the context clearly dictates otherwise. Thus, for example, the term “a member” is intended to mean a single member or a combination of members, “a material” is intended to mean one or more materials, or a combination thereof. Furthermore, the terms “proximal” and “distal” refer to the direction closer to and away from, respectively, an operator (e.g., surgeon, physician, nurse, technician, etc.) who would insert the medical implant and/or instruments into the patient. For example, the portion of a medical instrument first inserted inside the patient's body would be the distal portion, while the opposite portion of the medical device (e.g., the portion of the medical device closest to the operator) would be the proximal portion.

While the application has been illustrated and described in detail in the drawings and foregoing description, the same is to be considered as illustrative and not restrictive in character, it being understood that only the selected embodiments have been shown and described and that all changes, modifications and equivalents that come within the spirit of the invention as defined herein or by any of the following claims are desired to be protected.

What is claimed is:

1. An implant system, comprising:
at least a first anchor engageable to a first vertebra and a second anchor engageable to a second vertebra;
an elongate connecting member extending along an axis between a first end and an opposite second end, said connecting member including a length between said first and second ends sized to extend between and be engaged to said first and second anchors when said first and second anchors are engaged to the first and second vertebrae; and
an elongate fusion member extending between a first end and an opposite second end, said elongate fusion member being comprised of bone growth material and including a length between said first and second ends sized for positioning in contact with bony portions of the first and second vertebrae when the elongate fusion member is positioned alongside said connecting member.

2. The system of claim 1, wherein said connecting member is rigid to prevent motion of the first and second vertebrae when engaged to the first and second vertebrae with said first and second anchors, and said fusion member is offset laterally from said connecting member along a distal side of said connecting member.

3. The system of claim 1, wherein said first and second anchors each include a bone engaging portion engageable to respective ones of the first and second vertebrae and a receiver portion extending from said bone engaging portion, said receiving portions each defining a passage for receiving said connecting member therein.

4. The system of claim 3, wherein said elongate fusion member includes a body extending from said first end to said second end and engagement features extending outwardly from said body of said elongate fusion member, said engagement features directly engaging said bone engaging portion of each of said first and second anchors.

5. The system of claim 4, where said bone engaging portion is a threaded shaft of a bone screw.

6. The system of claim 1, wherein said elongate fusion member includes a body extending between said first and second ends, said body including deployable engaging features engaged thereto, said engaging features including a first position extending along said body and a second position projecting outwardly from said body for engaging the bony portions of the first and second vertebrae.

7. The system of claim 6, wherein said engagement features are comprised of shape memory material and deploy to said second position in response to a temperature change.

8. The system of claim 1, wherein said elongate fusion member includes a connector extending laterally from said second end of said fusion member toward said second end of said first elongate member, said second connector including an extension extending from said connection, said extension being axially aligned with connecting member and extending from said connector in a direction away from said second end of said connecting member.

9. An implant system, comprising:
at least a first anchor engageable to a first vertebra and a second anchor engageable to a second vertebra; and
an elongate stabilization member including a connection portion extending along a longitudinal axis between a first end and an opposite second end, said connection portion including a length between said first and second ends sized to extend between and be engaged to said first and second anchors when said first and second anchors are engaged to the first and second vertebrae, said elongate stabilization member further including a deployable fusion portion carried by said connection portion, said fusion portion including a first collapsed condition for insertion of said elongate stabilization member between said first and second anchors and a second deployed condition projecting outwardly from said connection portion sized to engage bony portions of the first and second vertebrae, said fusion portion including an interior with bone growth material in said interior in said deployed condition to facilitate fusion of the bony portions of the first and second vertebrae.

10. The system of claim 9, wherein said elongate stabilization member includes a lumen extending from said second end that is in fluid communication with said interior of said fusion portion.

11. The apparatus of claim 9, wherein:
said connection portion is rigid to prevent motion of the first and second vertebrae when engaged to the first and second vertebrae with said first and second anchors, and
said fusion portion includes a permeable bag carrying said bone growth material.

12. A method, comprising:
inserting through an incision in a patient an elongate connecting member between first and second anchors engaged to posterior elements of the first and second vertebrae;
engaging the connecting member to the first and second anchors;
inserting a fusion member through the incision to a location adjacent to the first and second anchors and the connecting member and positioning the fusion member in contact with bony portions of the first and second vertebrae; and
engaging the fusion member to at least one of the first and second anchors and transverse processes of the first and second vertebrae.

13. The method of claim 12, wherein:
inserting the connecting member includes guiding the connecting member along a minimally invasive insertion path that extends from the incision to the first and second anchors; and
inserting the fusion member includes guiding the fusion member along an insertion path that substantially follows the minimally invasive insertion path.

14. The method of claim 13, further comprising:
engaging a trailing end of the connecting member to a distal end of an inserter; and
guiding the connecting member along the minimally invasive insertion path with the inserter.

15. The method of claim 14, further comprising:
first and second elongated extensions extending proximally from the first and second anchors through skin of the patient to proximal end portions of the first and second extensions; and
the inserter includes a mounting portion pivotally mounted to the proximal end portions of the first and second elongated extensions and an elongate arm extending transversely to the mounting portion to the distal end of the inserter, wherein guiding the connecting member includes moving the elongate arm of the inserter into the patient along the minimally invasive insertion path.

16. The method of claim 15, further comprising:
mounting a trailing end of the fusion member to a distal end of a second inserter; and
guiding the fusion member along the insertion path with the second inserter.

17. The method of claim 16, further comprising:
maintaining the incision in an open condition with the elongate arm of the inserter to guide insertion of the fusion member and the second inserter into the patient along the insertion path.

18. The method of claim 15, further comprising:
mounting a trailing end of the fusion member to the distal end of the inserter; and
guiding the fusion member along the insertion path with the inserter.

19. The method of claim 12, wherein inserting the elongate member is rigid to prevent motion of the first and second vertebrae when engaged to the first and second anchors and the fusion member include bone growth material.

20. The method of claim 12, wherein inserting the fusion member includes positioning the fusion member along the connecting member in a location offset laterally from the connecting member and distally of the connecting member, and further comprising deploying engagement features of the fusion member to engage at least one of the first and second anchors and transverse processes of the first and second vertebrae.
